(19) United States
(12) Patent Application Publication
(10) Pub. No.: US 2010/0167396 A1
(43) Pub. Date: Jul. 1, 2010

(54) Application of Anti-Apoptotic Gene Expression in Mammalian Cells for Perfusion Culture

(75) Inventors: John Edward Murphy, Berkeley, CA (US); Konstantin Borislavov Konstantinov, Newton, MA (US); John Christopher Thrift, Benicia, CA (US); Tarangsri Nivitchanyong, Oakland, CA (US); Michael Betenbaugh, Baltimore, MD (US)

Correspondence Address: BAYER HEALTHCARE LLC, Law & Patents, 800 Dwight Way, BERKELEY, CA, 94710, US

(73) Assignees: BAYER HEALTHCARE LLC, Berkeley, CA (US); JOHNS HOPKINS UNIVERSITY, Baltimore, MD (US)

(21) Appl. No.: 12/297,620

(22) PCT Filed: Apr. 21, 2007
(86) PCT No.: PCT/US2007/009749

§ 371 Date: Mar. 19, 2010

Related U.S. Application Data
(60) Provisional application No. 60/793,905, filed on Apr. 21, 2006.

(51) Int. Cl. C12N 5/10

(52) U.S. Cl. 435/358; 435/375; 435/325; 435/352

(57) ABSTRACT

The present invention relates to preventing or delaying programmed cell death by expressing one or more anti-apoptotic polypeptides in a cell expressing recombinant Factor VIII such that programmed cell death in the cell is prevented or delayed. The present invention also relates to increasing production of recombinant Factor VIII by expressing one or more anti-apoptotic polypeptides in a cell such that production of recombinant Factor VIII by the cell is increased. Recombinant cells useful for producing Factor VIII.

This application claims benefit of U.S. Provisional Application Ser. No. 60/793,905; filed on Apr. 21, 2006, the contents of which are incorporated herein by reference in their entirety.

### FIELD OF THE INVENTION

The present invention relates to anti-apoptotic genes as a means for improving cell survival, cell physiology, and protein production. Specifically, the present invention relates to inhibiting programmed cell death in a cell line secreting recombinant factor VIII (FVIII) by expressing one or more anti-apoptotic polypeptides in the cell.

### BACKGROUND OF THE INVENTION

Mammalian cell culture is the system of choice for many recombinant protein production processes due to its ability to produce proteins with proper post-translational modifications. With manufacturing demand rising, there is a strong interest in improving process efficiency to increase product yield and quality. The modification of the apoptotic cell death pathway is one avenue available to achieve this goal. Apoptosis has been recognized as a major cause of death in cell culture, the result of culture insults such as nutrient and growth factor deprivation, oxygen depletion, toxin accumulation, and shear stress (Cotter and Al-Rubeai, Trends Biotechnol. 13(4):150-5, 1995; Mastrangelo, et al., Biotechnol. Bioeng. 67(5):544-54, 2000a; Mastrangelo, et al., Biotechnol. Bioeng. 67(5):544-54, 2000b; Mercille and Massie, Cytotechnology 15(1-3):117-28, 1994; Sanfeliu and Stephanopoulos, Biotechnol. Bioeng. 64(1):46-53, 1999; Lengwehasatit and Dickson, Biotechnol. Bioeng. 80(7):719-30, 2002; Zanghi, et al., Biotechnol. Bioeng. 64(1):108-19, 1999; Wong, et al., Biotechnol. Bioeng. 94(2):373-82, 2006). Apoptosis limits the maximum viable cell density, promotes the release of toxic metabolites from dead cells, and potentially decreases heterologous protein yield (Chiang and Sisk, Biotechnol. Bioeng. 91(7):779-92, 2005; Figueroa, et al., Biotechnol. Bioeng. 73(3):211-22, 2001; Figueroa, et al., Metab. Eng. 5(4):230-45, 2003; Figueroa, et al., Biotechnol. Bioeng. 85(6):589-600, 2004; Mastrangelo, et al., 2000a,b; Mercille and Massie, 1999).

To address these issues, there have been numerous reports investigating the expression of anti-apoptotic genes in small scale, batch, and perfusion cell culture processes (Chiang and Sisk, 2005; Mercille and Massie, 1999; Kim and Lee, Biotechnol. Bioeng. 71(3):184-93, 2000; Mastrangelo, et al., 2000a,b; Meents, et al., Biotechnol. Bioeng. 80(6):706-16, 2002; Tey, et al., J. Biotechnol. 79(2):147-59, 2000a; Tey, et al., Biotechnol. Bioeng. 68(1):31-43, 2000b; Vives, et al., Biotechnol. Prog. 19(1):84-9, 2003). In many of these studies, expression of the anti-apoptotic gene of interest increases viability of cultures exposed to toxic insults, nutrient deprivation, or toxin accumulation. Several of these studies have explored the use of anti-apoptotic genes from the Bcl-2 family (Singh, et al., Biotech. Bioeng. 52(1):166-75, 1996; Goswami, et al., Biotechnol. Bioeng. 62(6):632-40, 1999; Tey, et al., 2000a,b; Figueroa, et al., 2001; Meents, et al., 2002; Arden and Betenbaugh, Trends Biotechnol. 22(4):174-80, 2004; Sung, et al., Biotechnol. Prog. 21(1):50-7, 2005; Sauerwald, et al., Biotechnol. Bioeng. 94(2):362-72, 2006). Others have explored the use of functional viral homologs of Bcl-2, such as the adenovirus E1B-19K protein (Mercille, et al., Biotechnol. Bioeng. 63(5):516-28, 1999), the Kaposi sarcoma-associated virus KSBcl-2 protein, and the Epstein-Barr virus BHRF-1 protein (Vives, et al., 2003). Even though the sequence homology of Bcl-2 and E1B-19K is confined to certain conserved domains (Chiou, et al., J. Virol. 68(10):6553-66, 1994; Subramanian, et al., Oncogene 11(11):2403-9, 1995a), their functions are interchangeable in suppressing apoptosis resulting from adenovirus infection and adenovirus E1A protein expression (Rao, et al., Proc. Natl. Acad. Sci. USA 89(16):7742-6, 1992; Chiou, et al., 1994; Subramanian, et al., Cell Growth Differ. 6(2):131-7, 1995b). E1B-19K inhibits apoptosis by binding to pro-apoptotic proteins Bax, Bak, and Bik, and the p53 tumor suppressor protein (Boyd, et al., Oncogene 11(9):1921-8, 1995; Farrow, et al., Nature 374(6524):731-3, 1995; Han, et al., Mol. Cell Biol. 16(10):5857-64, 1996a; Han, et al., Genes Dev 10(4):461-77, 1996b; Lomonosova, et al., Oncogene 24(45):6796-808, 2005). During p53-induced apoptosis, E1B-19K binds Bak and inhibits Bax-Bak interaction, preventing release of the pro-apoptotic factors, cytochrome c and Smac/DIABLO, from the mitochondria (Henry, et al., Oncogene 21(5):748-60, 2002).

Aven is an anti-apoptotic protein, identified in a yeast two-hybrid screen (Chau, et al., Mol. Cell 6(1):31-40, 2000), which inhibits caspase activation. Following an external or internal apoptotic stimulus, cytochrome c is released from the mitochondrial intermembrane space, where it initiates Apaf-1 oligomerization, and this association recruits and activates caspase-9 (Saleh, et al., J. Biol. Chem. 274(25):17941-5,1999; Adams and Cory, Curr. Opin. Cell Biol. 14(6):715-20, 2002). The activated caspase-9 further activates downstream caspases leading to cellular degradation (Wolf and Green, J. Biol. Chem. 274(29):20049-52, 1999). Aven inhibits Apaf-1 self-association and therefore, suppresses the caspase-9 activation (Chau, et al., 2000). Aven binds to Bcl-x_{L} enhancing the anti-apoptotic property of Bcl-x_{L} following caspase-1 induced apoptosis (Chau, et al., 2000). In addition, Aven expression enhances the protective effect of Bcl-x_{L} in Chinese Hamster Ovary (CHO) cells exposed to various apoptotic insults including culture in spent medium and serum withdrawal (Figueroa, et al., 2004). Recently, CHO cells expressing Aven and E1B-19K were observed to grow to a higher cell density, survive longer, and generate higher levels of a monoclonal antibody in small-scale spinner flasks and large-scale fed-batch culture. The increase in volumetric productivity was due primarily to the enhanced cell viability provided by Aven and E1B-19K expression (Figueroa, et al., Biotechnol. Bioeng., Epub, 2006).

An alternative culture mode to batch and fed-batch processing for animal cells is perfusion culture. During perfusion culture, cells secreting recombinant protein are retained in the bioreactor while fresh nutrient media is continuously supplied and the conditioned media is continuously removed, along with the protein of interest and metabolic by-products. Unlike batch culture where accumulation of metabolic by-products and depletion of nutrients can limit the cell culture performance, perfusion culture establishes a steady-state environment that allows cells to be cultured for long periods at high density (Tolbert, et al., In Vitro 17(10):885-90, 1981; Butler, et al., J. Cell Sci. 61:351-63, 1983; Prior, et al., J. Parenter Sci. Technol. 43(1):15-23, 1989; Ozturk, et al., Biotechnol. Bioeng. 48(3):201-206, 1995; Michaels, et al., European Society for Animal Cell Technology (ESACT) Conference, 1999). Nonetheless, a reduction in cell viability has still been observed in perfusion culture with apoptosis being identified as a major cause of death, particularly at high cell density and low perfusion rate conditions (Al-Rubeai, et al., Cytotechnology 9(1-3):85-97, 1992; Mercille, et al., 1994; Banik and Heath, Appl. Biochem. Biotechnol. 61(3):211-29, 1996; Bierau, et al., J. Biotechnol. 62(3):195-207,1998; Thrift, et al., European Society for Animal Cell Technology (ESACT) Conference, 2003). There have been studies investigating apoptosis inhibition in perfusion culture by expressing anti-apoptotic proteins such as Bcl-2 (Bierau, et al., 1998; Fassnacht, et al., Cytotechnology 30:95-105,1998; Tey, et al., Apoptosis 9(6):843-52, 2004) and E1B-19K (Mercille and Massie, Biotechnol. Bioeng. 63(5):529-43, 1999) in cells secreting a recombinant protein. These reports both demonstrate an improvement in cell viability and an increase in viable cell density of the cell culture expressing the anti-apoptotic genes in comparison to the parental cell culture. However, the effects on the specific productivity have been conflicting. Bierau, et al., report a decrease in specific monoclonal antibody productivity in a Bcl-2 expressing hybridoma cell line cultured in spin filter and ultrasonic filter perfusion bioreactors. Another study with a Bcl-2 expressing hybridoma cell line cultured in a fixed bed reactor shows an increase in specific monoclonal antibody productivity (Fassnacht, et al., 1999). The NS0 myeloma cell line expressing E1B-19K shows a higher specific productivity of the chimeric antibody (Mercille and Massie, 1999), but another NS0 cell line expressing Bcl-2 presents lower specific antibody productivity (Tey, et al., 2004). These previous studies, however, examined cell viability and productivity over a limited number of dilution rates and with changing cell densities in concert with the perfusion rates. The feeding strategy in one study was designed such that the cells accumulate over time and the perfusion rate (volume of medium per volume of cell culture per day) increased corresponding to the rising cell density (Tey, et al., 2004). In another study, the culture was performed at a constant VVD (volume of fresh medium per effective cell suspension volume per day), while the cell density increases to a plateau at a later stage (Mercille and Massie, 1999).

The various approaches described above to increase protein productivity have succeeded to varying degrees. Nevertheless, there is still a need to develop methods to increase production of cell-related product, for example, recombinant proteins, especially in large-scale commercial production. In addition, there is a need to develop methods for prevention or delaying programmed cell death.

### SUMMARY OF THE INVENTION

An object of the invention is to provide a method for preventing or delaying programmed cell death in a cell line secreting recombinant FVIII by expressing one or more anti-apoptotic polypeptides in the cell. The method includes expressing or inducing the expression of one or more anti-apoptotic polypeptides, for example, Aven or E1B-19K, in the cell such that programmed cell death in the cell is prevented or delayed.

It is another object of the invention to provide a method of increasing production of a cell-related product by a recombinant cell, for example, a cell secreting recombinant FVIII. The method includes expressing or inducing the expression of one or more anti-apoptotic polypeptides, for example, Aven or E1B-19K, in the cell such that production of the cell-related product by the cell is increased.

Another object of the invention is to provide a method of increasing production of a recombinant cell, for example, a cell secreting recombinant FVIII. The method includes expressing or inducing the expression of one or more anti-apoptotic polypeptides, for example, Aven or E1B-19K, in the cell such that production of the recombinant cell is increased.

Another object of the invention is to provide a method of increasing production of a recombinant cell, for example, a cell expressing recombinant FVIII, in a small scale, batch, or perfusion cell culture process. In addition, an object of the invention is to provide a method of increasing production of a recombinant cell, for example, a cell expressing recombinant FVIII, in a large scale bioreactor or culture device of a commercial production. Another object of the invention is to provide a method of increasing production of a recombinant cell, for example, a cell expressing recombinant FVIII, in a large scale perfusion cell culture bioreactor or perfusion cell culture device of a commercial production.

Another object of the invention is to provide a recombinant cell useful for producing cell-related product, for example, a cell line secreting recombinant FVIII. The recombinant cell expresses or can be induced to express at least two anti-apoptotic polypeptides.

### DESCRIPTION OF THE FIGURES

FIG. 1A depicts the immunoblot results of five E1B-19K cell lines to confirm E1B-19K expression and six Aven-E1B-19K cell lines to confirm Aven and E1B-19K expression.

FIG. 1B depicts the viability of E1B-19K cell lines compared to the blank vector and parental cell lines after seeding at 5×10⁶ cells/mL in shake flask suspension culture. Each data point represented sixteen cell count with standard deviation of 5% or smaller.

FIG. 1C depicts the viability of Aven-E1B-19K cell lines compared to the blank vector and parental cell lines after seeding at 5×10⁶ cells/mL in shake flask culture. Each data point represented sixteen cell count with standard deviation of 5% or smaller.

FIG. 1D depicts the comparison of average viabilities of E1B-19K and Aven-E1B-19K cell lines after seeding at 5×10⁶ cells/mL in shake flask culture. The Aven-E1B-19K cell lines showed the most improvement in average viability followed by the E1B-19K cell lines, with the blank vector and parental cell lines as controls.

FIG. 2A depicts the percentage of caspase-3 activated cells at day 2 after seeding at 5×10⁶ cells/mL in shake flask culture.

FIG. 2B depicts the percentage of Annexin-V positive cells (Annexin-V positive 7-AAD negative) at day 2 after seeding at 5×10⁶ cells/mL in shake flask culture. The viable, apoptotic, and dead cells were differentiated using Annexin-V and 7-AAD staining.

FIG. 3A depicts the correlation between Aven expression level and percent viability of Aven-E1B-19K cell lines at day 6 after seeding at 5×10⁶ cells/mL in shake flask culture. Aven expression level was quantified from the immunoblot in FIG. 1A.

FIG. 3B depicts the correlation between E1B-19K expression level and percent viability of Aven-E1B-19K and E1B-19K cell lines at day 6 after seeding at 5×10⁶ cells/mL in shake flask culture. The Aven and E1B-19K expression level was quantified from the immunoblots in FIG. 1A.

FIG. 3C depicts the correlation between E1B-19K expression level and percent viability, similar to FIG. 2B but including the high E1B-19K expression clones (#6, #11).

FIG. 4A depicts the parental, blank vector, E1B-19K #6, and Aven-E1B-19K#22 cell lines untreated, treated with same volume of DMSO used to deliver thapsigargin, or treated with 2 μM thapsigargin. Viability after 48 hours treatment was assessed. Each value is an average viability of three (3) cell culture flasks with the same treatment.

FIG. 4B depicts the parental, blank vector, E1B-19K #6, and Aven-E1B-19K #22 cell lines untreated, treated with same volume of DMSO used to deliver thapsigargin, or treated with 2 μM thapsigargin. Percentage of caspase-3 activated cells after 24 hours of treatment was evaluated.

FIG. 5A depicts the cell line performance when cultured in optimal conditions by passaging every two days and monitored for growth rate and productivity. Normalized growth rate of the cell lines was calculated using the value of the parental cell line as 100%. The growth rate was calculated from the viable cell density at the time of passage (every two days).

FIG. 5B depicts the cell line performance when cultured in optimal conditions by passaging every two days and monitored for growth rate and productivity. Normalized specific productivity of the cell lines was calculated using the value of the parental cell line as 100%. The specific productivity was determined using the secreted FVIII amount in the culture as measured by the chromogenic assay.

FIG. 6A depicts the time profile of the cell culture in a 12-L bioreactor operating at a stepwise decline in perfusion rates. Viable cell density (open square) and percent viability (close triangle) of the parental culture.

FIG. 6B depicts the profile of the cell culture in a 12-L bioreactor operating at a stepwise decline in perfusion rates. Percentage of caspase-3 activated cells of the parental culture.

FIG. 6C depicts the profile of the cell culture in a 12-L bioreactor operating at a stepwise decline in perfusion rates. Viable cell density (open square) and percent viability (close triangle) of the Aven-E1B-19K culture.

FIG. 6D depicts the profile of the cell culture in a 12-L bioreactor operating at a stepwise decline in perfusion rates. Percentage of caspase-3 activated cells of the Aven-E1B-19K culture.

FIG. 7A depicts the comparison between the parental and the Aven-E1B-19K culture in a 12-L bioreactor operating at a stepwise decline in perfusion rate. Specific productivity relative to the value of the parental culture at 0.5 perfusion rate.

FIG. 7B depicts the comparison between the parental and the Aven-E1B-19K culture in a 12-L bioreactor operating at a stepwise decline in perfusion rate. Specific productivity plotted as a percentage of the initial level for each culture.

FIG. 7C depicts the comparison between the parental and the Aven-E1B-19K culture in a 12-L bioreactor operating at a stepwise decline in perfusion rate. Growth rate relative to the parental value at 0.5 perfusion rate.

FIG. 7D depicts the comparison between the parental and the Aven-E1B-19K culture in a 12-L bioreactor operating at a stepwise decline in perfusion rate. Growth rate as a percentage of the initial growth rate. The value is an average calculated from daily measurements of the culture operating at the respective perfusion rate.

FIG. 8A depicts the comparison between the parental and the Aven-E1B-19K culture during a stepwise decline in perfusion rate: specific glucose consumption rate.

FIG. 8B depicts the comparison between the parental and the Aven-E1B-19K culture during a stepwise decline in perfusion rate: specific glutamine consumption rate.

FIG. 8C depicts the comparison between the parental and the Aven-E1B-19K culture during a stepwise decline in perfusion rate: specific lactate production rate.

FIG. 8D depicts the comparison between the parental and the Aven-E1B-19K culture during a stepwise decline in perfusion rate: specific oxygen uptake rate as a percentage of the initial level.

FIG. 8E depicts the comparison between the parental and the Aven-E1B-19K culture during a stepwise decline in perfusion rate: glucose level in the bioreactor.

FIG. 8F depicts the comparison between the parental and the Aven-E1B-19K culture during a stepwise decline in perfusion rate: glutamine level in the bioreactor.

FIG. 9 depicts the average cell diameter of the parental and the Aven-E1B-19K culture running at decreasing perfusion rates.

### DETAILED DESCRIPTION OF THE INVENTION

The present invention relates to the prevention or delaying programmed cell death in a recombinant cell. The present invention provides methods to preventing or delaying programmed cell death in a recombinant cell by expressing one or more anti-apoptotic polypeptides in the cell. The present invention also provides methods to increase production of a cell-related product in a recombinant cell by expression one or more anti-apoptotic polypeptides in the cell. In addition, the present invention provides recombinant cells useful for producing cell-related product or cellular therapy.

The present invention relates to the prevention or delaying programmed cell death in a recombinant cell secreting recombinant FVIII. The present invention provides methods to preventing or delaying programmed cell death in a recombinant cell secreting recombinant FVIII by expressing one or more anti-apoptotic polypeptides, for example, Aven or E1B-19K, in the cell. The present invention also provides methods to increase production of a recombinant cell secreting recombinant FVIII by expression one or more anti-apoptotic polypeptides, for example, Aven or E1B-19K, in the cell. In addition, the present invention provides recombinant cells useful for producing cell-related product or cellular therapy.

The genetic manipulation techniques may include standard recombinant DNA techniques in which the target gene of interest is integrated into the mammalian genome or an extra-chromosomal element in order to allow expression of the integrated gene as a heterologous protein. The technology described in this application may be appropriate for mammalian and other eukaryotic cell lines for which apoptosis occurs during the cell culture process. These cell lines may be obtained from sources such as American Type Culture Collection (ATCC). Such technology may be appropriate for any eukaryotic cells that undergo programmed cell death and can be genetically manipulated to generate heterologous proteins of interest such as those listed above.

Expression of one or more anti-apoptotic polypeptides in a cell may be achieved by any suitable means known to one skilled in the art. For example, one may introduce one or more polynucleotide constructs encoding one or more anti-apoptotic polypeptides. Such constructs may be expression constructs and may include at least one inducible promoter operably linked to the polynucleotide encoding one or more anti-apoptotic polypeptides.

According to the present invention, one or more anti-apoptotic polypeptide may be expressed. Anti-apoptotic polypeptide includes any polypeptides having an activity of inhibiting or decreasing apoptosis in a cell. For example, an anti-apoptotic polypeptide may be encoded by heterologous polynucleotides, such as genes in eukaryotic cells or viruses. In one embodiment, an anti-apoptotic polypeptide is Aven or E1B-19K.

In a recombinant cell, anti-apoptotic polypeptide may be expressed alone or in combination with others. For example, Aven may be expressed alone or co-expressed with E1B-19K. One or more expression constructs may be used to express two or more anti-apoptotic polypeptides.

Recombinant cells of the present invention include any suitable cells known and available to one skilled in the art. In one embodiment, the recombinant cells are Baby Hamster Kidney (BHK) or CHO cells. In addition, methods of the present invention may be applied to recombinant cells in a large-scale bioreactor or culture device of commercial production.

The present invention also provides recombinant cells useful for producing cell-related product. These cells express or may be induced to express one or more anti-apoptotic polypeptides. In one embodiment, these cells express at least two anti-apoptotic polypeptides.

In order that this invention may be better understood, the following examples are set forth. These examples are for the purpose of illustration only, and are not to be construed as limiting the scope of the invention in any manner. All publications mentioned herein are incorporated by reference in their entirety.

### EXAMPLES

The expression of FVIII in a BHK-producing cell line engineered to express the E1B-19K gene alone and in combination with the Aven gene was examined. The cell lines were analyzed initially in small-scale shake flask cultures for viability and product yield. Subsequently, the behavior of a cell line expressing both Aven and E1B-19K was compared to the parental cell line in perfusion culture running at a range of specific perfusion rates (volume of media per viable cell per day). The viable cell density was maintained at a constant level while the specific perfusion rate was decreased step-wise, from high rates that allow rapid turnover of nutrients to low rates in which the nutrient medium was fed much more slowly and spent culture medium removed more slowly in concert. The effect of varying the perfusion rate on viability, caspase-3 activation level, nutrient consumption, metabolite production, and protein productivity was evaluated and compared between a parental cell line and cell line expressing Aven and E1B-19K.

A perfusion system may consist of a bioreactor for cell culture and a settler for cell retention. With the inclined settler, viable cells are separated from the conditioned media and returned to the bioreactor (Batt, et al., Biotechnol. Prog. 6(6):458-64, 1990; Searles, et al., Biotechnol. Prog. 10(2):198-206, 1994). Fresh media is continuously supplied to the bioreactor to maintain the culture volume. Cell culture is removed at an adjusted purge rate to sustain the cell density in the bioreactor. Such a bioreactor set-up enables the examination of the relationship between specific perfusion rate and apoptosis in culture and allows the elucidation of any possible benefits of cell engineering with anti-apoptotic genes on the perfusion strategy.

### Example 1

### Plasmid Construction

A vector, pBUDCE4.1 (pBUD) (Invitrogen, Carlsbad, Calif.), was used for constitutive expression of Aven (SEQ ID NO: 1-2) and E1B-19K (SEQ ID NO: 3-6). The vector was designed for constitutive expression of two genes simultaneously, using the pCMV promoter and the pEF-1alpha promoter. The Aven gene was subcloned into pBUDCE4.1 vector using BamHI site and expressed by the CMV promoter. The E1B-19K gene was subcloned into the same pBUDCE4.1 vector using NotI and XhoI sites and expressed by the EF-1 alpha promoter. The Aven-E1B-19K vector contains each gene expressed by the corresponding promoter. The E1B-19K vector only contains the E1B-19K gene expressed by the EF-1 alpha promoter. The blank vector refers to the original pBUDCE4.1 vector.

### Example 2

### Creation of Stable Cell Lines

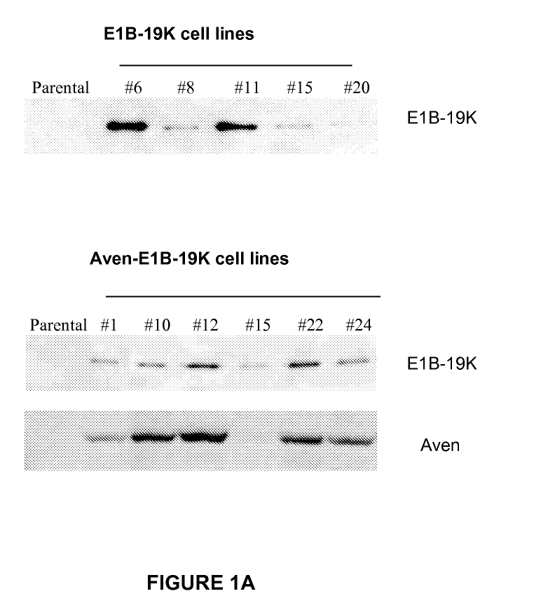

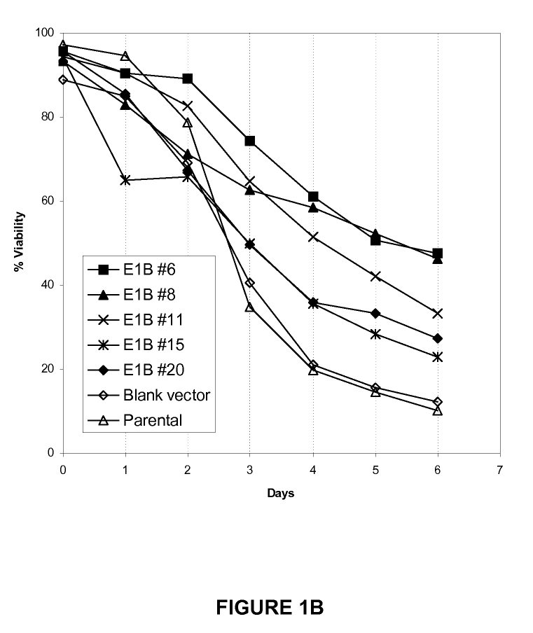

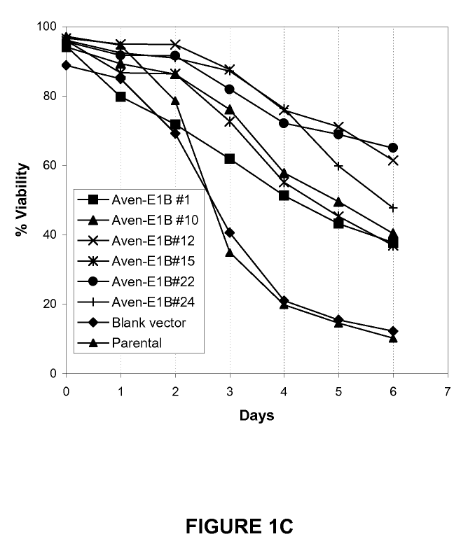

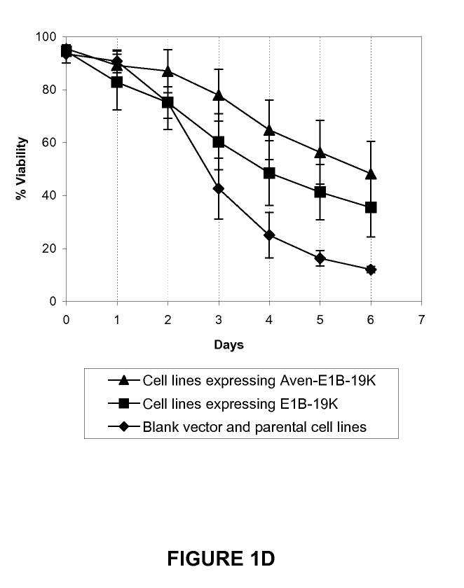

A BHK-21 cell line expressing recombinant FVIII (BHK-FVIII) was supertransfected with blank vector, E1B-19K vector, or Aven-E1B-19K vector using Lipofectamine Plus (Invitrogen, Carlsbad, Calif.) according to the manufacturer's instructions. Stable cell lines were created under selection of 1 mg/mL Zeocin (Invitrogen) in adherent culture supplemented with 5% FBS. Approximately 100 clones of each construct were isolated and analyzed for FVIII expression levels (SEQ ID NO: 7). Approximately 25 clonal isolates were selected, and the expression of Aven and E1B-19K expression was detected by immunoblotting. Based on the expression level of Aven and/or E1B-19K and FVIII productivity, 3-6 clones of each construct were adapted to serum-free suspension culture in the absence of Zeocin. Aven and E1B-19K expression from cells cultured with no antibiotics was confirmed to be stable by another round of immunoblotting (FIG. 1A).

### Example 3

### Immunoblotting Analysis

Cells were collected with lysis buffer containing 1% NP-40, 120 mM Tris-HCl, 150 mM NaCl, 0.2 mM PMSF, and 1 mM EDTA. Protein concentration was determined using BCA protein assay kit (Pierce, Rockford, Ill). Equal amounts of protein were loaded per lane. The proteins were separated by gel electrophoresis, transferred to a nitrocellulose membrane (Bio-Rad, Hercules, Calif.), and immunoblotted with rabbit anti-Aven antibody at a dilution of 1:1000 (Chau, et al., 2000) and mouse anti-E1B-19K antibody at a dilution of 1:40 (Calbiochem, San Diego, Calif.).

### Example 4

### Shake Flask Culture Analysis and Apoptosis Detection

Cells were seeded at a density of 5×10⁶ cells/mL in shake flasks and cultured in an incubator maintained at 37° C. and 5% CO₂. The culture was examined every 24 hours for viability, caspase-3 activation, Annexin-V binding assay, and nutrient measurements. Cell count was performed by trypan blue exclusion assay using a Cedex cell counting device (Innovatis, Bielefeld, Germany). Caspase-3 activation was detected using a Guava PCA system (Guava Technologies, Hayward, Calif.). Cells were washed with cold PBS, fixed with Cytofix/Cytoperm solution (BD Pharmingen, San Diego, Calif.), and then stained with PE-conjugated monoclonal active caspase-3 antibody (BD Pharmingen) before analyzing with the Guava system. For the Annexin-V binding assay, cells were collected and incubated using a Guava Nexin kit and analyzed on the Guava PCA system according to manufacturer's instructions (Guava Technologies). Levels of glutamine, glutamate, glucose, and lactate were measured by the YSI analyzer (YSI Life Sciences, Yellow Springs, Ohio).

### Example 5

### FVIII Quantitation

Factor VIII was quantified using the chromogenic assay kit (Chromogenix, Milano, Italy) and the coagulation assay. In the chromogenic assay, FVIII quantity was determined by the factor stimulation of the activation of factor X to factor Xa. The reaction was performed with factor X and factor IXa provided in excess. The activated factor Xa cleaved a chromogenic substrate resulting in a color change. The absorbance at 405 nm was measured, and FVIII activity was determined against a standard curve. The coagulation assay measured FVIII activity from the rate of fibrin clot development after the sample was added to the FVIII-deficient plasma. The reaction was facilitated by an activated partial thromboplastin reagent and CaCl₂.

### Example 6

### Bioreactor Evaluation

Two 12-L bioreactors were inoculated with the parental cell line or the Aven-E1B-19K cell line at a density of 1×10⁶ cells/mL. The cells were accumulated until the cell density reached 20×10⁶ cells/mL, then the cell density was maintained constant at 20×10⁶ cells/mL by adjusting the cell discard rate. The dissolved oxygen concentration was maintained at 50% air saturation. The pH was controlled at a set point of 6.8, and the temperature was maintained at 35.5° C. Cell viability, viable cell density, and cell diameter were measured by the Cedex cell counting device (Innovatis, Bielefeld, Germany). Levels of glutamine, glutamate, glucose, and lactate were measured by the YSI analyzer (YSI Life Sciences, Yellow Springs, Ohio). The oxygen uptake rate was calculated as a percentage of its initial value at 0.5 perfusion rate which was assumed to be 100%. The relative oxygen uptake rate was determined from the difference in partial pressure of the oxygen in the silicone tubing and in the reactor, while assuming that the mass transfer rate and area (k_{L}a) value, the reactor volume, and the average viable cell density remained constant over the course of the culture.

### Example 7

### Cell Viability in High Cell Density Shake Flask Cultures

Five cell lines expressing E1B-19K, six cell lines expressing Aven-E1B-19K, a blank vector cell line, and the parental cell line were seeded into shake flasks at high density (5×10⁶ cells/mL). As shown in FIGS. 1B and 1C, the viabilities of the cell lines expressing E1B-19K and Aven-E1B-19K were compared against the control parental and blank vector cell lines. After six days, the viability of the best performing E1B-19K clone (#6) was at 48%, the best performing Aven-E1B-19K clone (#22) was 65% viable. The viability of the blank vector cell line was 12% and that of the parental cell line was at 10%. Some variability in the viability measurements of each clonal isolate at the different time points was observed. The cell lines expressing anti-apoptotic genes maintained generally higher viabilities compared to the two control cell lines. To confirm that the construct of E1B-19K and Aven-E1B-19K had an effect on cell viability, average cell viabilities and standard deviations of the cell lines were determined and plotted (FIG. 1D). The BHK-FVIII cells expressing E1B-19K maintained viability on average 18 percentage points above the controls at day 3 and 23 percentage points above the controls at day 6. The average of the BHK-FVIII cells expressing both Aven and E1B-19K presented viability levels that were even higher: 35 percentage points on average above the controls at day 3, and 36 percentage points above the controls at day 6. The expression of E1B-19K improved cell viability relative to the control cell lines, and the combination of Aven and E1B-19K expression further improved the cell survival for batch cell cultures seeded at high density.

### Example 8

### Apoptosis in Shake Flask Culture

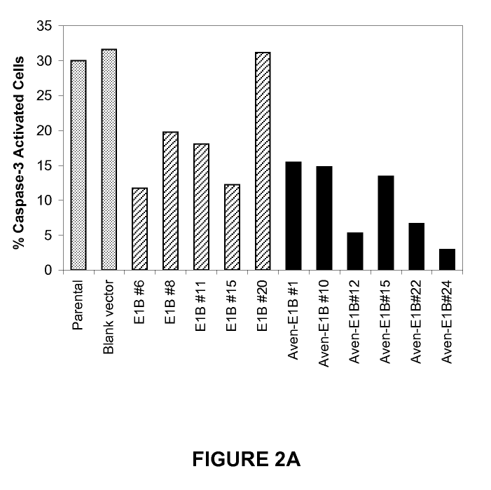

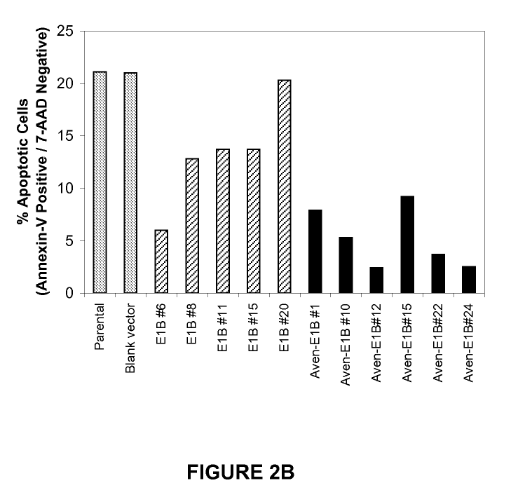

Cells were analyzed for caspase-3 activation and Annexin-V surface staining in order to examine the mode of cell death. Activation of caspase-3 is a major event in the apoptosis cascade leading to “a point of no return” that leads to activation of other caspases (Adams, Genes Dev. 17(20):2481-95, 2003; Danial and Korsmeyer, Cell 116(2):205-19, 2004). The caspase-3 activation and the Annexin-V assays were performed at day 2 when the viability varied from 66% to 89%, and before a large number of dead cells became apparent. As shown in FIG. 2A, all but one of the E1B-19K cell lines (#6, #8, #11, #15) and all of the Aven-E1B-19K cell lines showed lower percentages of caspase-3 activated cells than the parent or blank vector clone.

At an early stage in the apoptosis cascade, phosphatidylserine is translocated from the inner leaflet to the outer leaflet of the plasma membrane. Annexin-V has high binding affinity to phosphatidylserine and therefore, binds to the apoptotic cells. The loss of membrane integrity in dead cells exposes the phosphatidylserine in the inner leaflet allowing Annexin-V binding as well (Van Engeland, et al., Cytometry 31(1):1-9, 1998). The use of a membrane permeable DNA stain such as 7-AAD distinguishes apoptotic cells from dead cells. The viable, apoptotic, and dead cells were differentiated using Annexin-V and 7-AAD staining. As shown in FIG. 2B, the percentage of Annexin-V positive cells that were also 7-AAD negative was lower in E1B-19K cell lines #6, #8, #11, and #15 and in all Aven-E1B-19K cell lines. As with the caspase-3 assay, a higher percentage of Annexin-V positive/7-AAD negative cells were found in the parental, blank vector, and E1B-19K cell line #20. The E1B-19K #20 cell line expressed a very low level of E1B-19K (FIG. 1A), but some improvement in cell viability was observed in high cell density culture (FIG. 1B). The percentage of apoptotic cells was lower at day 2 in most of the Aven-E1B-19K clones as compared to the majority of the E1B-19K clones. This decrease in apoptotic cells correlated with the improvement in viability of the Aven-E1B-19K clones relative to the E1B-19K clones in FIG. 1D.

### Example 9

### Correlation Between Cell Viability and Expression Level

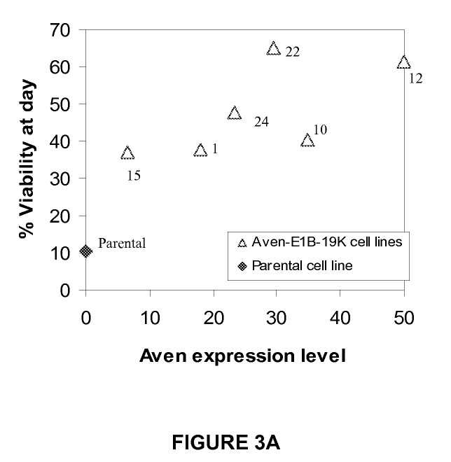

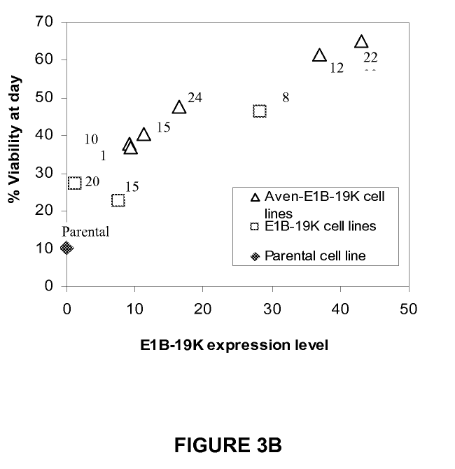

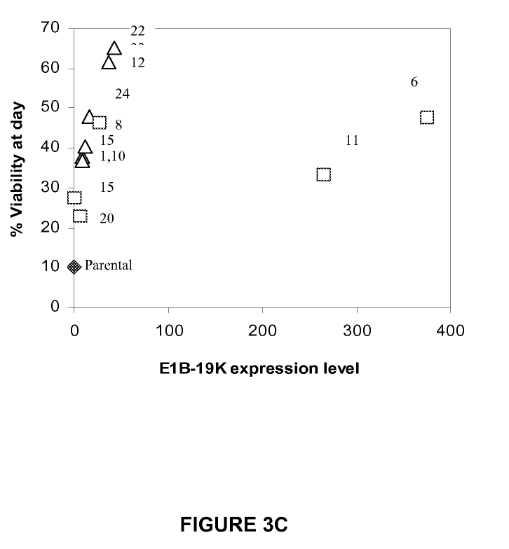

The relative expression level Aven and E1B-19K was estimated from the immunoblot band intensity measured using a densitometer (FIG. 1A), and plotted against the cell viability at day 6 of the high cell density shake flask (shown in FIGS. 1B and 1C). Six Aven-E1B-19K cell lines were examined for a relationship between cell viability and Aven expression. As shown in FIG. 3A, there was no clear correlation between percent viability and expression level of Aven. Aven-E1B-19K cell lines #12 and #22 included high viability and higher expression levels, but Aven-E1B-19K clone #10 had a lower viability despite the high Aven expression level. The relationship between percent viability and expression levels of E1B-19K is shown in FIGS. 3B and 3C. The E1B-19K expression level was relatively proportional to the percent viability of E1B-19K and Aven-E1B-19K cell lines at day 6 for these cell lines expressing moderate E1B-19K levels (FIG. 3B). The cell lines that presented high viability were found to express increased levels of E1B-19K (Aven-E1B #22, Aven-E1B #12, E1B-19K #8) and the cell lines with limited protection against cell death exhibited lower expression levels. The two E1B-19K cell lines (#6 and #11) with very high expression levels did not present any significant improvement in survival at day 6 of the high density cell culture (E1B-19K clone #6, 48% viable; E1B-19K clone #11, 33% viable) relative to the clones with expression levels multiple times lower and exhibited lower viabilities than some of the clones expressing both Aven and E1B-19K (FIG. 3C).

### Example 10

### Apoptosis Induced by Thapsigargin

Recombinant FVIII in mammalian cells has been shown to have low efficiency of secretion (Kaufman, et al., J. Biol. Chem. 263(13):6352-62, 1988). The majority of the intracellular FVIII was found in the endoplasmic reticulum (ER) in complexes with protein chaperones including BiP (Dorner, et al., J. Cell. Biol. 105(6 Pt 1):2665-74,1987), calnexin, and calreticulin (Pipe, et al., J. Biol. Chem. 273(14):8537-44, 1998). Thapsigargin is a chemical that disrupts the intracellular calcium homeostasis by inhibiting calcium uptake into the ER (Lytton, et al., J. Biol. Chem. 266(26):17067-71, 1991). Treatment of cells with thapsigargin induces ER stress and caspase-12 activation resulting in apoptosis (Nakagawa, et al., Nature 403(6765):98-103, 2000).

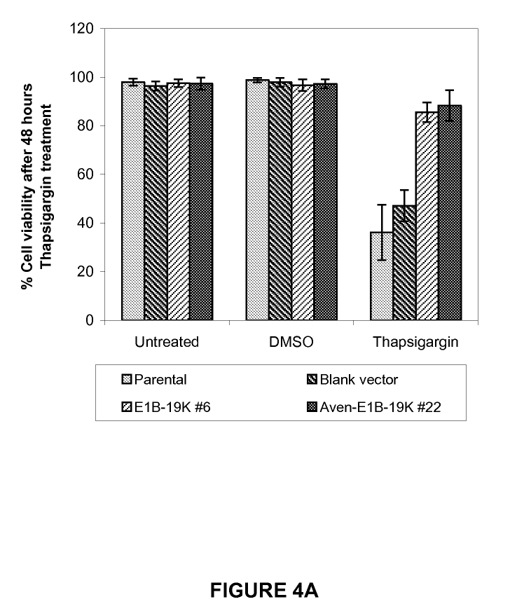

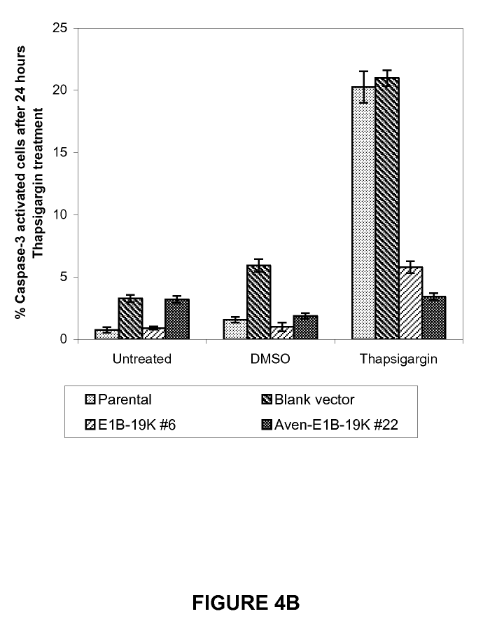

The engineered cell lines Aven-E1B-19K #22 and E1B-19K #6, in addition to the blank vector and the parental cell lines were treated with thapsigargin in shake flask culture. After 48 hours of treatment with 2 μM thapsigargin solution in DMSO, the controls (blank vector and parental) exhibited much lower viabilities than those observed for E1B-19K #6 and Aven-E1B-19K #22 (FIG. 4A). The viabilities of E1B-19K #6 and Aven-E1B-19K #22 were above 85% and the two control viabilities were below 50%. As a negative control, the untreated cells or those treated with DMSO did not exhibit significant loss in viability for any cell line. To examine apoptosis induction, caspase-3 activation was evaluated 24 hours after thapsigargin treatment. Approximately 20% of the control cell lines exhibited caspase-3 activation and less than 5% of the anti-apoptosis engineered cell lines showed caspase-3 activation (FIG. 4B). Untreated and DMSO-treated cells displayed lower levels of caspase-3 activity indicating that thapsigargin treatment was responsible for stimulation of caspase-3 (FIG. 4B). There was no difference in viability at the 24-hour time point even though the caspase-3 activity showed significant differences between control cells and cell lines expressing the anti-apoptotic genes.

### Example 11

### Expression in Serial Passage Culture

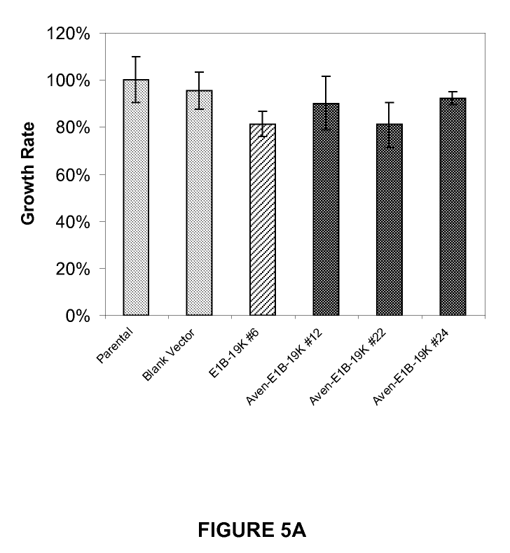

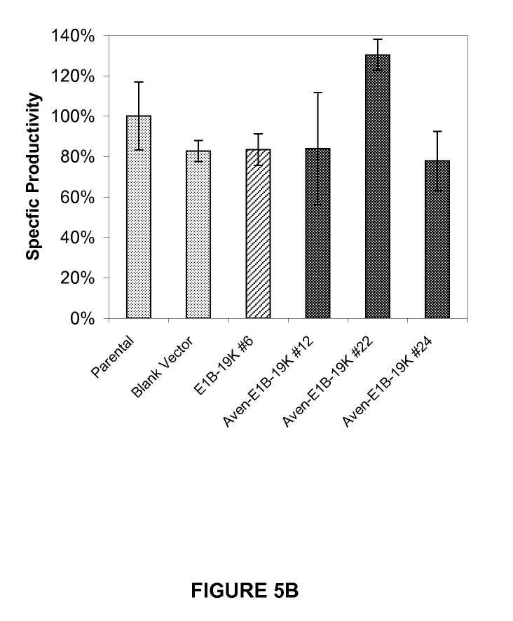

The cell lines were cultured in optimal conditions to evaluate cellular performance. A cell line was selected that maintained a high growth rate and had specific productivity comparable to the parental cell line. The cells were passaged every two days in shake flask suspension culture and monitored for growth rate and FVIII productivity. Cell lines that had maintained higher viabilities in high cell density batch culture were evaluated (Aven-E1B-19K #12, Aven-E1B-19K #22, Aven-E1B-19K #24, and E1B-19K #6). The growth rate of these cell lines was reduced slightly to between 81% and 92% of the parent (FIG. 5A). The specific productivity of these clones was found to vary between 83% and 130% of the parent (FIG. 5B). While most of the engineered clonal isolates had slightly lower productivity than the parental cell line, the Aven-E1B-19K #22 cell line produced approximately 30% more FVIII than the parent cell lines on a per cell basis. From these small-scale studies, the Aven-E1B-19K cell line #22 was selected as the final clone due to high viability in high cell density culture, slightly reduced growth rate (81%), and superior productivity to the parental cell line (130%).

### Example 12

### Co-expression on Viability and Apoptosis in Perfusion Culture

The parental BHK-FVIII cell line and the BHK-FVIII Aven-E1B-19K #22 cell line were evaluated in a 12-L continuous perfusion bioreactor. The bioreactor was inoculated and the cells were accumulated until a cell density of 2×10⁷cells/mL was achieved at which point density was maintained by purging cells from the system. The specific perfusion rate was set initially at 0.5 ηL/cell/day and then lowered in the following order: 0.5, 0.3, 0.2, and 0.15, followed by an increase back to 0.5 in order to observe whether or not the cells were able to recover after operation at low perfusion rates. Samples were taken from the bioreactor each day to determine cell viability, apoptosis status, nutrient and metabolite levels, and FVIII concentration. Other parameters including dissolved oxygen, temperature, and media composition were kept constant.

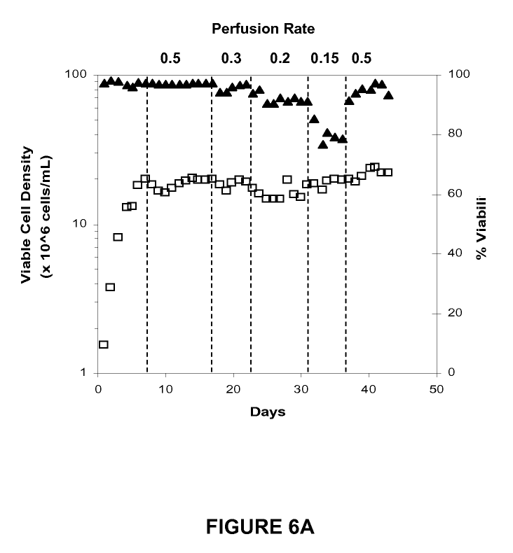

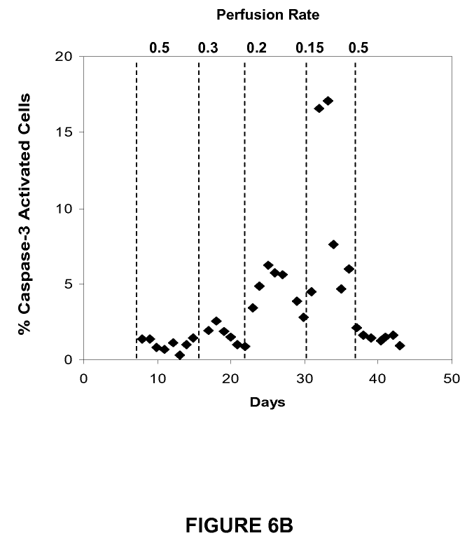

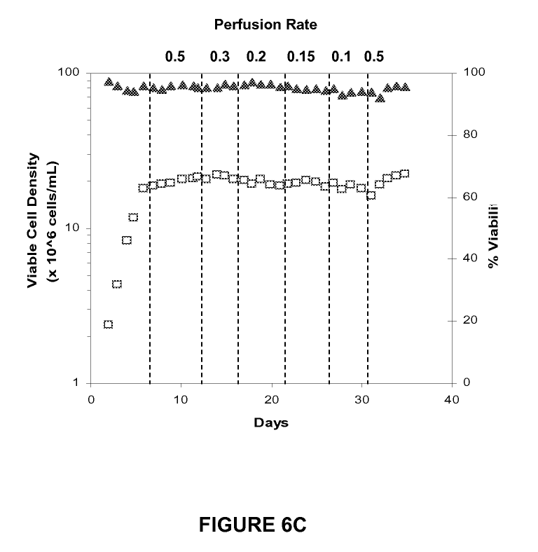

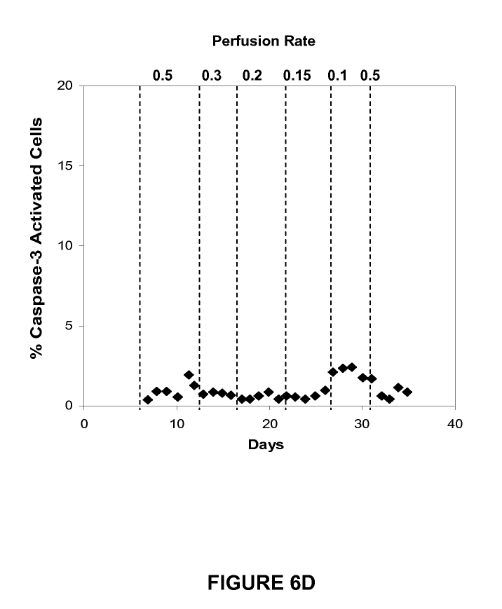

FIG. 6A shows the time profile of the viable cell density and viability of the parental cell culture. The cell density increased over the first seven days as the cells were accumulating in the bioreactor. A viable cell density of 2×10⁷ cells/mL was reached on day 7 at which time the culture mode was switched from cell accumulation to pseudo steady-state. The pseudo steady-state was achieved by bleeding the culture from the bioreactor at an adjusted rate in order to maintain the viable cell density at a constant level of 2×10⁷ cells/mL. The viability of the cells in the bioreactor remained relatively constant at 97% during the initial operation at 0.5 ηL/cell/day specific perfusion rate. At day 17, the perfusion rate was reduced to 0.3, and the viability of the parental cell line remained between 94% and 97% during this period. When the perfusion rate was lowered again to 0.2 at day 23, the average viability decreased to 91% over the next 7-day period of operation at a 0.2 perfusion rate. When the perfusion rate was lowered even further to 0.15, the viability declined relatively steadily to approximately 80%. At day 37, the perfusion rate was returned to 0.5 and the cell viability recovered to 94% at the end of the culture period. To examine whether or not apoptosis was present, caspase-3 activation assays were performed daily (FIG. 6B). The percentage of caspase-3 activated cells was at baseline during the initial specific perfusion rate of 0.5 and only slightly higher at the perfusion rate of 0.3. The percentage increased to 5% when the perfusion rate was at 0.2, which corresponds to a decrease in average cell viability from 94% to 91%. At the 0.15 perfusion rate, the percentage of caspase-3 activated cells increased rapidly to show activation above 15% during the same period when the cell viability was steadily dropping. Annexin-V staining was performed and showed a similar profile to the caspase-3 activation pattern. The decrease in cell viability at low perfusion rate was due, at least in part, to cells undergoing apoptosis.

Aven-E1B-19K cells were grown to a similar cell density (2×10⁷ cell/mL) and subjected to a similar stepwise decline in perfusion rate (FIGS. 6C and 6D). Although the operating conditions were similar, the viability profile of the Aven-E1B-19K culture was considerably different than that observed for the parental cells. The Aven-E1B-19K cell viability remained nearly constant at about 94% over the entire range of perfusion rates (FIG. 6C). When the perfusion rate was reduced to 0.15 ηL/cell/day, no reduction in viability was observed, remaining constant at about 94% as compared to an average of 80% for the parental cultures. Since the cell viability did not decrease with the Aven/E1B-19K cell line at the planned lowest specific perfusion rate of 0.15, the specific perfusion rate was further reduced from 0.15 to 0.1 on day 27 and ran at this minimal rate for four days. Even at this low perfusion rate, the cell viability remained elevated at an average of 94% over the entire 4-day period. In contrast, the perfusion of the parental cell culture could not be reduced below a level of 0.15 due to the danger of a complete loss of viable cells from culture. Apoptosis analysis was also performed on the Aven-E1B-19K culture throughout the culture period and showed that the cells maintained a basal level of caspase-3 activation (FIG. 6D) for most of the culture period. There was a slight but sustained elevation in the percentage of caspase-3 active cells to an average of 2% for the Aven-E1B-19K cells operating at the 0.10 perfusion rate. The percentage of Annexin-V positive cells showed similar low activation levels at these perfusion rates. The Aven-E1B-19K cell line maintained high viability and did not undergo significant levels of apoptosis even when cultured at the very low specific perfusion rates shown to negatively impact the parental cell line.

### Example 13

### Co-expression on FVIII Specific Productivity and Growth Rate in Perfusion Culture

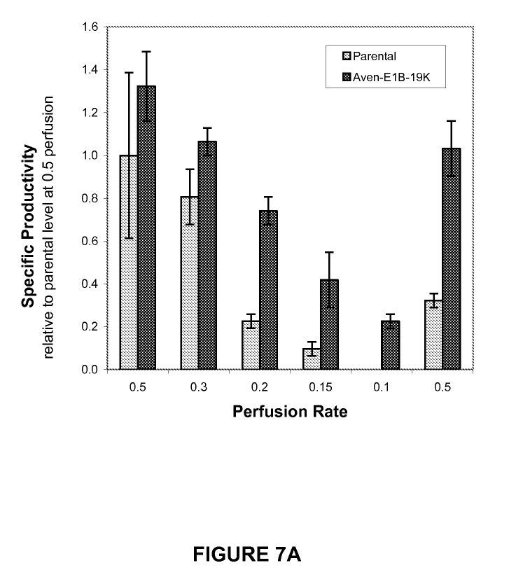

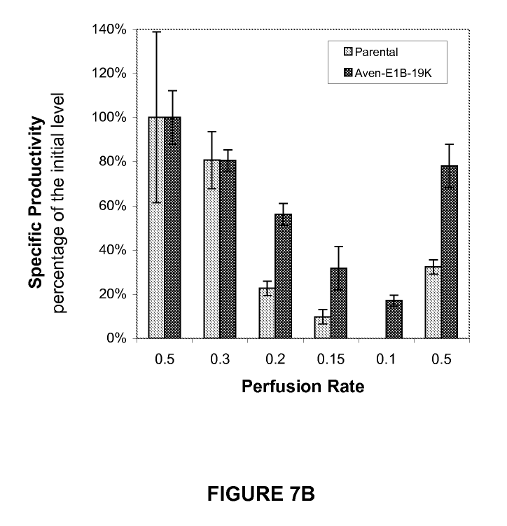

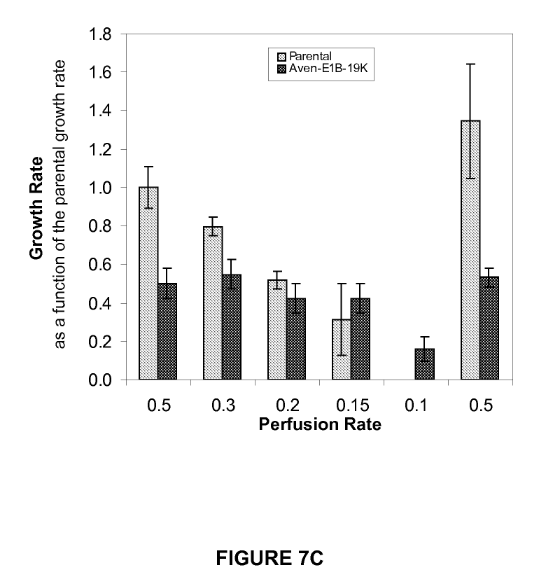

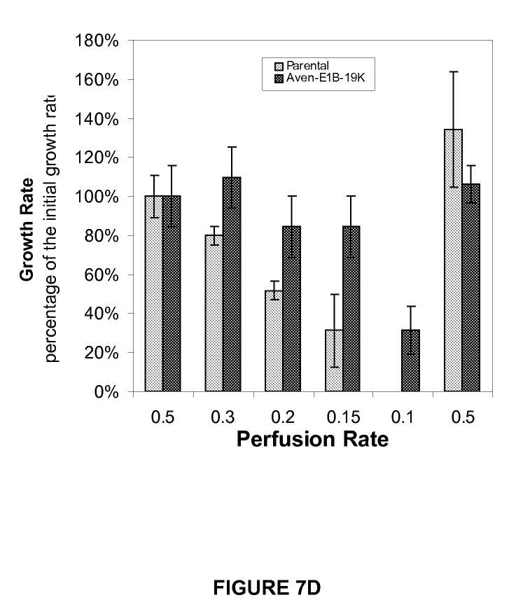

To compare the productivity of the parent and Aven-E1B-19K #22 cell lines in a perfusion bioreactor environment, the levels of FVIII were measured at the different perfusion rates. Shown in FIG. 7A is the specific productivity on a per cell basis for the two cell lines as a function of the FVIII productivity of the parental control at 0.5 ηL/cell/day perfusion rate. Shown in FIG. 7B is the reduction in productivity for each culture with decreasing perfusion rates relative to the 100% value of each culture at 0.5 perfusion rate. Both cultures exhibited a decline in productivity as the perfusion rates were progressively lowered. At all specific perfusion rates, the productivity of the Aven-E1B-19K #22 cell line was higher (see FIG. 4B). When the productivity was examined as a percentage of the initial level (FIG. 7B), the effect of expressing Aven and E1B-19K was evident. Following a perfusion rate reduction from 0.5 to 0.3, the specific productivity of both the parent and the Aven-E1B-19K culture declined from 100% to 80%. When the specific perfusion rate was reduced to 0.2 and 0.15, the decline in productivity was significantly more precipitous for the parental cell line. The parental productivity declined to approximately 23% of its initial value at 0.2 and approximate 10% at 0.1 perfusion rates, and the Aven-E1B-19K #22 cell line maintained 56% and 32% of its initial productivity level over that same interval. During the recovery period (0.5 perfusion rate at the end), the parental productivity only recovered to 37% of its original value, reflecting the amount of damage to parental cell performance caused by a severe reduction in perfusion of fresh nutrients. In contrast, the Aven-E1B-19K cell line was able to recover to 80% of the initial productivity.

Another outcome of reducing the specific perfusion rate was a reduction of growth rate. FIG. 7C shows the cellular specific growth rates plotted as a function of the parental culture running at 0.5 specific perfusion rate. FIG. 7D shows the growth rates as a percentage of the initial growth rate for both parental and Aven-E1B-19K cell line. The specific growth rate of the Aven-E1B-19K cell line at a perfusion rate of 0.5 was approximately half of the growth rate of the parent cell line for that same perfusion rate (FIG. 7C). As the perfusion rate declined, the growth rate of the parental cells declined progressively as well (FIG. 7D). The growth rate of the parental cells decreased from 100% to 80%, 52%, and 31% following each reduction in perfusion rate. This decrease in growth rates was expected as lower perfusion rates reduced nutrient availability. The parental cell growth rate was at its highest, 134% of the beginning value, when the perfusion rate was increased to 0.5 after being reduced to 0.15 (FIG. 7D). In contrast, the Aven-E1B-19K cell line exhibited a much different growth rate pattern as the perfusion rate was lowered. The growth rate of the Aven-E1B-19K culture changed slightly from 100% to 109%, 84%, and 84% at the lower perfusion rates of 0.3, 0.2, 0.15, and decreased sharply to 31% at perfusion rate of 0.1 (FIG. 7D). At a perfusion rate of 0.15, the growth rate of the Aven-E1B-19K cell line was still at 84% of its value at 0.5 perfusion rate while the parental control growth rate was only 30% of the value at 0.5 perfusion rate. The growth rate of the Aven-E1B-19K cell line did increase once again when the perfusion rate was returned to 0.5, but its level was much closer to its original growth rate than that observed for the parental control (FIG. 7D).

### Example 14

### Co-expression on Nutrient Metabolism in Perfusion Culture

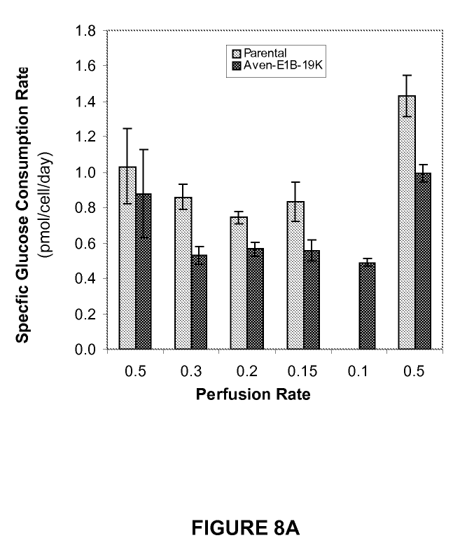

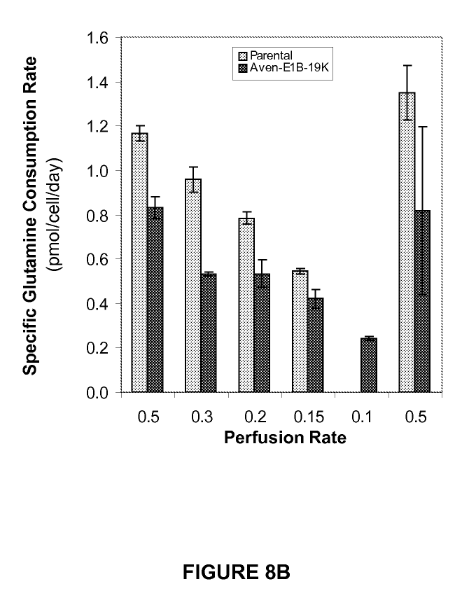

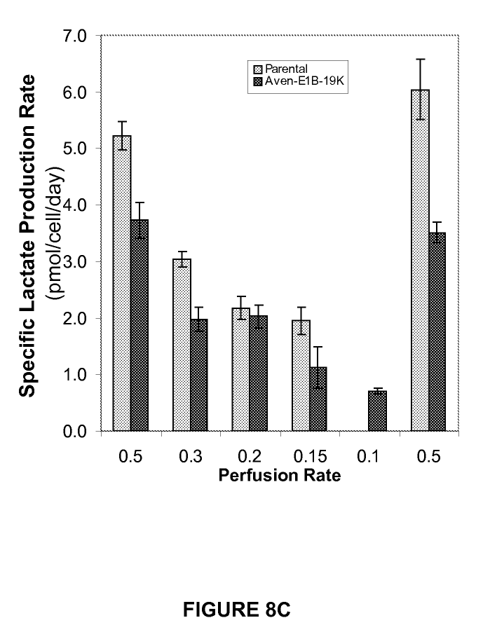

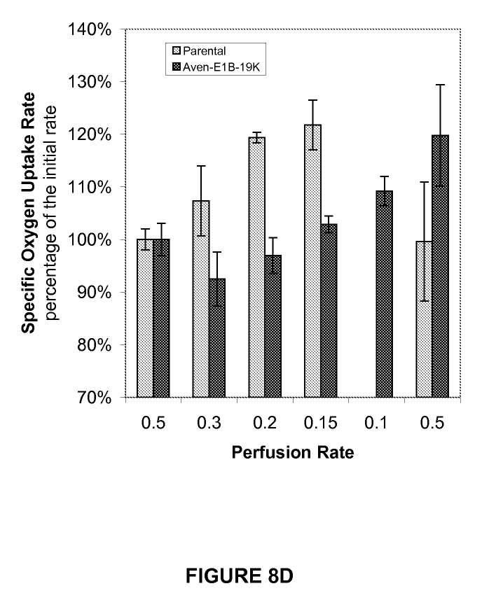

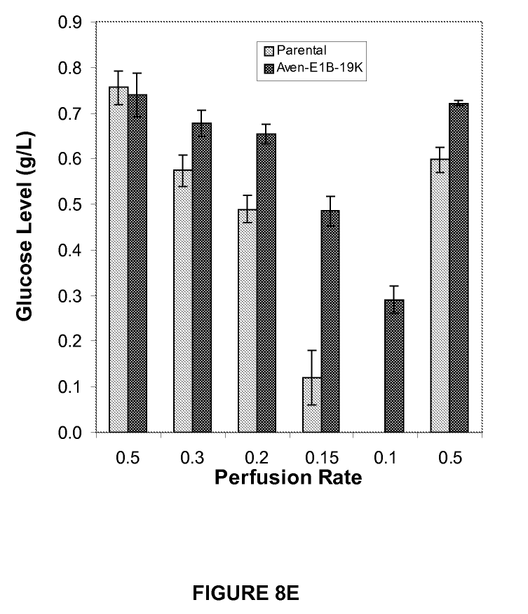

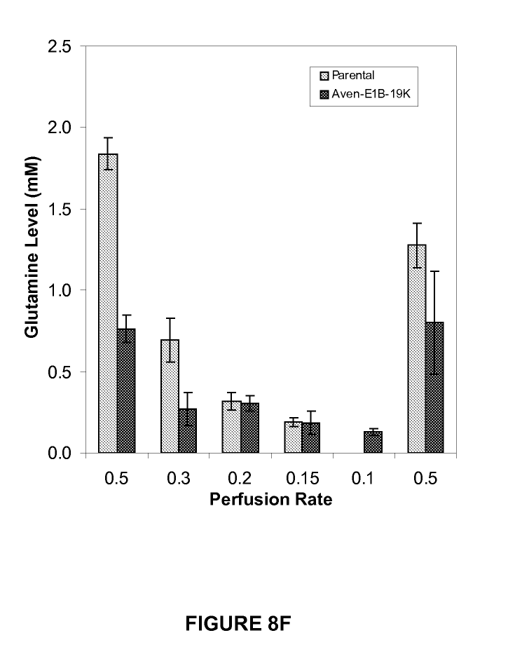

To examine the effect of cell metabolism at different perfusion rates on nutrient and metabolite levels, glutamine, glucose, dissolved oxygen, and lactate levels were measured each day in the bioreactor. Specific consumption and production rates (amount of nutrient consumed or produced per cell per day) are shown in FIGS. 8A-8D. Glucose and glutamine levels in the bioreactor are shown in FIGS. 8E and 8F. As observed with the growth rates, glucose (FIG. 8A) and glutamine (FIG. 8B) consumption rates of the parental cell line tended to decline with decreasing perfusion rate and then rebound to levels above the initial value during the 0.5 recovery period. The glucose available in the bioreactor was not limiting in either case (FIG. 8E); the glutamine available was low at the lower perfusion rates (0.3, 0.2, 0.15, and 0.1) for both parental and Aven-E1B-19K cell lines (FIG. 8F). Across all perfusion rates, the Aven-E1B-19K cell line had consistently lower specific glucose and glutamine consumption rates than the parental (FIG. 8A and 8B). This decrease in specific nutrient consumption rate has been observed previously in perfusion culture for cell lines expressing E1B-19K (Mercille and Massie, 1999) and Bcl-2 (Bierau, et al., 1998). In addition, the decrease of glutamine consumption at progressively lower perfusion rates was less apparent for the Aven-E1B-19K cell line as compared to the parent. The glutamine consumption level of the Aven-E1B-19K cell line at 0.15 perfusion rate was 24% of its value at 0.5, while the glutamine consumption of the parental cell line at 0.15 was 10% of its value at 0.5. The parental cell line exhibited a much sharper increase in glucose and glutamine consumption upon returning the perfusion rate to 0.5 at the end of the culture.

The specific lactate production rates were lower for the Aven-E1B-19K cell line (FIG. 8C) which suggests that these cells produced less glycolytic metabolites on a per cell basis as well as consuming less nutrients than the parental cell line at the different perfusion rates. The lactate production rate decreased with lower perfusion rate for both parental and Aven-E1B-19K cell line, but the decline was not as severe for the Aven-E1B-19K cell line. Unlike the glucose and glutamine consumption trends, the oxygen uptake rates of the parental cell line increased from 100% to 122% with the decreasing perfusion rate from 0.5 to 0.15, and the Aven-E1B-19K oxygen uptake rates remained relatively steady from 92% to 103% (FIG. 8D). When the perfusion rate was decreased to 0.1 in the Aven-E1B-19K culture, the oxygen uptake rate did increase.

### Example 15

### Cell Size

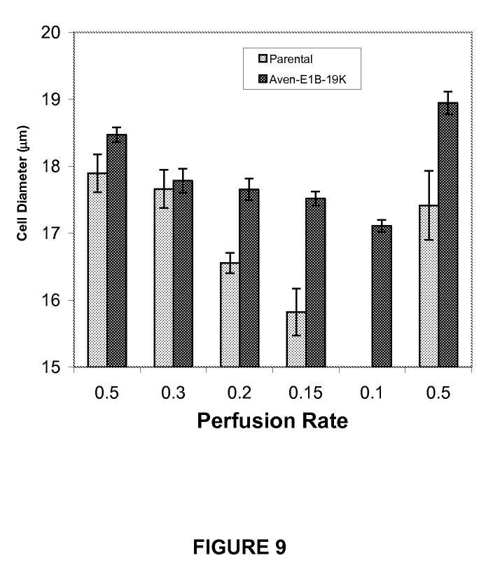

FIG. 9 shows the average cell diameters of the parental and the Aven-E1B-19K cell lines in the bioreactor running at different perfusion rates. Cell size of the parental cell line decreased from 17.9 μm at 0.5 perfusion rate to 17.7, 16.6, and 15.8 μm at 0.3, 0.2, and 0.15 perfusion rates, respectively. This decrease correlates with the reduced viability at lower perfusion rates (FIG. 6A). The loss of cell volume or cell shrinkage is one of the major hallmarks of apoptosis (Bortner and Cidlowski, Cell Death Differ. 9(12):1307-10, 2002); therefore, this decrease in cell size may relate to apoptosis induction. In contrast, the Aven-E1B-19K cell line showed a smaller decrease in cell size from 18.5 μm at 0.5 perfusion rate to 17.8, 17.7, 17.5, and 17.1 μm at 0.3, 0.2, 0.15, and 0.1 perfusion rates, respectively. The cell diameter of parental and Aven-E1B-19K cell lines increased to initial level or higher when the perfusion rate was finally increased to 0.5.

The expression of E1B-19K delayed the BHK-FVIII cells from undergoing apoptosis in high cell density shake flask culture, and co-expression of Aven and E1B-19K enhanced the protection even further relative to both the parental and a blank vector cell line. Thus, the expression of the anti-apoptotic genes allows for more efficient survival and protein production at much lower perfusion rates. These effects provide benefits that can lead to more robust and productive cell lines for perfusion cell culture applications in biotechnology.

All publications and patents mentioned in the above specification are incorporated herein by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A method for preventing or delaying programmed cell death in a cell expressing recombinant Factor VIII comprising expressing one or more anti-apoptotic polypeptides in the cell such that programmed cell death in the cell is prevented or delayed.

2. The method of claim 1, wherein expression of one or more anti-apoptotic polypeptides is controlled by at least one inducible heterologous promoter operably linked to a polynucleotide encoding said one or more anti-apoptotic polypeptides.

3. canceled

4. The method of claim 1, wherein the anti-apoptotic polypeptides are selected from the group consisting of Aven and E1B-19K.

5. canceled

6. canceled

7. canceled

8. canceled

9. The method of claim 1, wherein the recombinant cell is a mammalian cell.

10. canceled

11. The method of claim 9, wherein the recombinant cell is a CHO cell or a BHK cell.

12. canceled

13. The method of claim 1, wherein the recombinant cell is in a small scale cell culture process, batch cell culture process, perfusion cell culture process, a large scale bioreactor, culture device of a commercial production, a large scale perfusion cell culture bioreactor, or perfusion cell culture device of a commercial production.

14. canceled

15. canceled

16. A method of increasing production of Factor VIII by a recombinant cell comprising expressing one or more anti-apoptotic polypeptides in the cell such that production of Factor VIII by the cell is increased.

17. The method of claim 16, wherein expression of one or more anti-apoptotic polypeptides is controlled by at least one inducible heterologous promoter operably linked to a polynucleotide encoding said one or more anti-apoptotic polypeptides.

18. canceled

19. The method of claim 16, wherein the anti-apoptotic polypeptides are selected from the group consisting of Aven and E1B-19K.

20. canceled

21. canceled

22. canceled

23. canceled

24. The method of claim 1, wherein the recombinant cell is a mammalian cell.

25. canceled

26. The method of claim 24, wherein the recombinant cell is a CHO cell or a BHK cell.

27. canceled

28. The method of claim 16, wherein the recombinant cell is in a small scale cell culture process, batch cell culture process, perfusion cell culture process, a large scale bioreactor, culture device of a commercial production, a large scale perfusion cell culture bioreactor, or perfusion cell culture device of a commercial production.

29. canceled

30. canceled

31. canceled

32. A recombinant cell useful for producing Factor VIII expressing or capable of expressing at least two anti-apoptotic polypeptides.

33. canceled

34. The recombinant cell of claim 32, wherein the anti-apoptotic polypeptides are selected from the group consisting of Aven and E1B-19K.

35. The recombinant cell of claim 34, wherein expression of one or more anti-apoptotic polypeptides is controlled by at least one inducible heterologous promoter operably linked to a polynucleotide encoding said one or more anti-apoptotic polypeptides.

36. The recombinant cell of claim 32, wherein the recombinant cell is a mammalian cell.

37. canceled

38. The recombinant cell of claim 36, wherein the recombinant cell is a CHO cell or a BHK cell.

39. canceled

40. The recombinant cell of claim 32, wherein the cell produces Factor VIII in a small scale cell culture process, batch cell culture process, perfusion cell culture process, a large scale bioreactor, culture device of a commercial production, a large scale perfusion cell culture bioreactor, or perfusion cell culture device of a commercial production.

41. canceled

42. canceled
